(19) United States
(12) Patent Application Publication
(10) Pub. No.: US 2017/0128555 A9
(43) Pub. Date: May 11, 2017

(54) PLACENTAL COMPOSITIONS FOR STIMULATION OF IMMUNITY TO PD-L1

(71) Applicant: Batu Biologics, Inc., San Diego, CA (US)

(72) Inventors: Samuel C. Wagner, San Diego, CA (US); Thomas E. Ichim, San Diego, CA (US)

(73) Assignee: Batu Biologics, Inc., San Diego, CA (US)

(21) Appl. No.: 15/043,422

(22) Filed: Feb. 12, 2016

Related U.S. Application Data
(63) Continuation-in-part of application No. PCT/US2015/028710, filed on May 1, 2015.
(60) Provisional application No. 61/987,657, filed on May 2, 2014.

(51) Int. Cl. A61K 39/00

(52) U.S. Cl. CPC A61K 39/0005; A61K 2039/515; A61K 2039/585

(57) ABSTRACT

Disclosed is the new, useful, and unexpected finding that immunization to placental endothelial cells stimulated with interferon gamma result in antibodies to the checkpoint inhibitor PD-L1. In one embodiment, the invention teaches the use of ValloVax™ to induce immunological hyperresponsiveness and reduction of costimulatory need for T cell activation. In another embodiment the invention teaches means of selecting placental populations for enhanced expression of PD-L1 in order to augment immunity towards checkpoint inhibitors.

### CROSS-REFERENCE TO RELATED APPLICATIONS

This application is a continuation-in-part of PCT Application No. PCT/US15/028710 filed on May 1, 2015, which claims the benefit of U.S. Provisional Application No. 61/987,657 filed on May 2, 2014, the contents of which are incorporated herein by reference in their entirety.

### BACKGROUND OF THE INVENTION

### Field of the Invention

The invention relates to the field of cancer vaccines, more specifically, the invention relates to stimulation of immunity to checkpoint inhibitors, more specifically the invention relates to stimulation of antibodies to PD-L1 through vaccination with endothelial cells, more specifically the invention relates to endothelial cells derived from placenta that have been pretreated with interferon gamma.

The immune system is comprised of multiple different cell types, biologically active compounds and molecules and organs. These include lymphocytes, monocytes and polymorphonuclear leukocytes, numerous soluble chemical mediators (cytokines and growth factors), the thymus, postnatal bone marrow, lymph nodes, liver and spleen. All of these components work together through a complex communication system to fight against microbial invaders such as bacteria, viruses, fungi and parasites, and tumor cells. Together, these components recognize specific molecular antigens as foreign or otherwise threatening, and initiate an immune response against cells or viruses that contain the foreign antigen. The immune system also functions to eliminate damaged or cancerous cells through active surveillance using the same mechanisms used to recognize microbial or viral invaders. The immune system recognizes the damaged or cancerous cells via antigens that are not strictly foreign, but are aberrantly expressed or mutated in the targeted cells.

Unfortunately, while immunity to cancer cells has been demonstrated, this is not effective at a level sufficient to induce clinical responses in many cases. One method of augmenting immune response is to depress the self-regulatory mechanisms that the immune responses uses to regulate itself. Inhibition of inhibitory signals, called “checkpoint inhibitors” have demonstrated promising clinical efficacy in numerous situations.

A clinical study reported by Herbst et al. was designed to evaluate the single-agent safety, activity and associated biomarkers of PD-L1 inhibition using the MPDL3280A, a humanized monoclonal anti-PD-L1 antibody administered by intravenous infusion every 3 weeks (q3w) to patients with locally advanced or metastatic solid tumors or leukemias. Across multiple cancer types, responses as per RECIST v1.1 were observed in patients with tumors expressing relatively high levels of PD-L1, particularly when PD-L1 was expressed by tumor-infiltrating immune cells. Specimens were scored as immunohistochemistry 0, 1, 2, or 3 if <1%, ≧1% but <5%, ≧5% but <10%, or ≧10% of cells per area were PD-L1 positive, respectively. In the 175 efficacy-evaluable patients, confirmed objective responses were observed in 32 of 175 (18%), 11 of 53 (21%), 11 of 43 (26%), 7 of 56 (13%) and 3 of 23 (13%) of patients with all tumor types, non-small cell lung cancer (NSCLC), melanoma, renal cell carcinoma and other tumors (including colorectal cancer, gastric cancer, and head and neck squamous cell carcinoma). Interestingly, a striking correlation of response to MPDL3280A treatment and tumor-infiltrating immune cell PD-L1 expression was observed. In summary, 83% of NSCLC patients with a tumor-infiltrating immune cell IHC score of 3 responded to treatment, whereas 43% of those with IHC 2 only achieved disease stabilization. In contrast, most progressing patients showed a lack of PD-L1 upregulation by either tumor cells or tumor-infiltrating immune cells.

In another study examining the MPDL3280A antibody, Powles et al, treated patients with metastatic urothelial bladder cancer. Responses were often rapid and many occurring at the time of the first response assessment (6 weeks). This study also confirmed that tumors expressing PD-L1-positive tumor-infiltrating immune cells had particularly high response rates. A response rate of 43% (95% CI: 26-63%) achieved in advanced UBC patients with PD-L1 IHC 2/3 tumors provides evidence of noteworthy clinical activity of MPDL3280A. Patients with PD-L1 IHC 0/1 tumors had a response rate of only 11% (95% CI: 4-26%).

Immunization to break tolerance to self antigens has previously been used therapeutically in a variety of contexts, unfortunately, to date, this has only been used to induce immunity using synthetic sources of checkpoint inhibitors. The advantage of utilizing a naturally-occurring source of PD-L1 is overcoming restricted repertoire of antibodies elicited, thus acting to generated increased antibody complentary determining region diversity, and therefore wider applicability.

### BRIEF DESCRIPTION OF THE DRAWINGS

FIG. 1A illustrates the stimulation of anti-PD-1L antibody by ValloVax™ Vaccination in Patient 1.

FIG. 1B illustrates the stimulation of anti-PD-1L antibody by ValloVax™ Vaccination in Patient 2.

FIG. 1C illustrates the stimulation of anti-PD-1L antibody by ValloVax™ Vaccination in Patient 3.

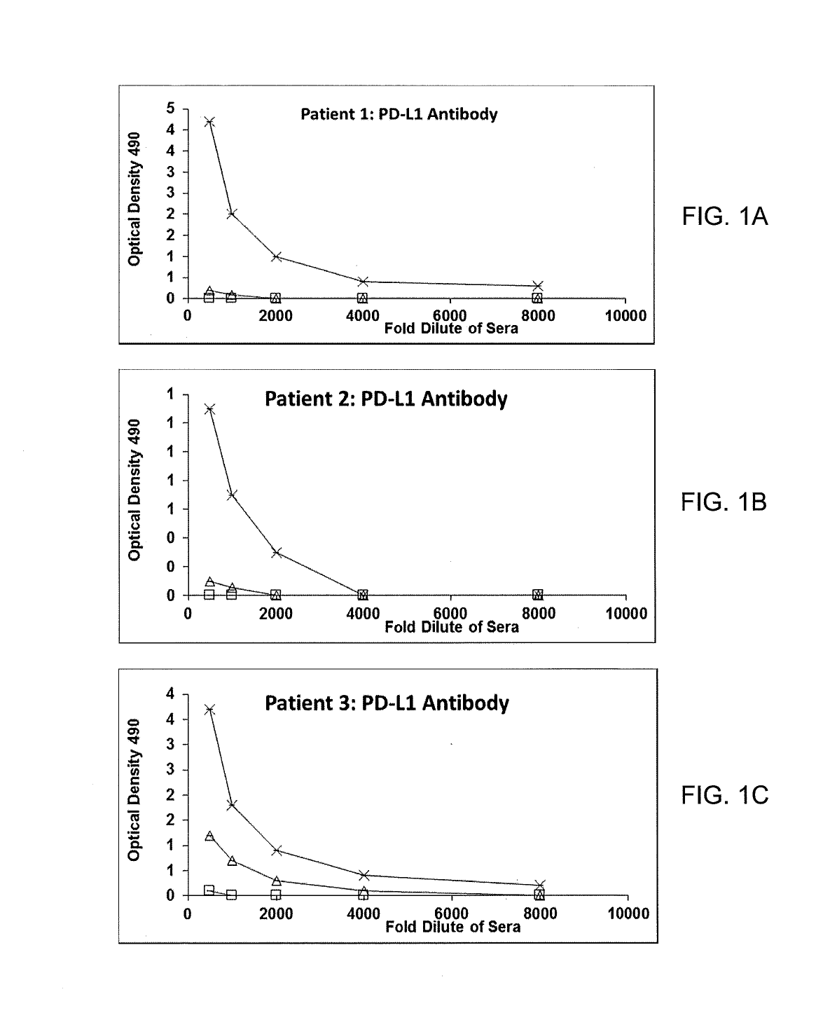

### DETAILED DESCRIPTION OF THE INVENTION

The invention teaches the utilization of placental vaccination to elicit antibody responses to the checkpoint inhibitor PD-L1. Specifically, the invention teaches that protocols in use for immunization with ValloVax™, and derivatives thereof, are useful in the induction of immunity towards PD-L1. Additionally, the invention provides means of upregulating PD-L1 expression including culture with TGF-beta, IL-10, VEGF, and PGE-2.

Below are definitions useful for the practice of the invention:

“antigen-presenting cells” or “APCs” are used to refer to autologous cells that express MHC Class I and/or Class II molecules that present antigens to T cells. Examples of antigen-presenting cells include, e.g., professional or non-professional antigen processing and presenting cells. Examples of professional APCs include, e.g., B cells, whole spleen cells, monocytes, macrophages, dendritic cells, fibroblasts or non-fractionated peripheral blood mononuclear cells (PMBC). Examples of hematopoietic APCs include dendritic cells, B cells and macrophages. Of course, it is understood that one of skill in the art will recognize that other antigen-presenting cells may be useful in the invention and that the invention is not limited to the exemplary cell types described herein. APCs may be “loaded” with an antigen that is pulsed, or loaded, with antigenic peptide or recombinant peptide derived from one or more antigens. In one embodiment, a peptide is the antigen and is generally antigenic fragment capable of inducing an immune response that is characterized by the activation of helper T cells, cytolytic T lymphocytes (cytolytic T cells or CTLs) that are directed against a malignancy or infection by a mammal. In one, embodiment the peptide includes one or more peptide fragments of an antigen that are presented by class I MHC or class II MHC molecules. The skilled artisan will recognize that peptides or protein fragments that are one or more fragments of other antigens may used with the present invention and that the invention is not limited to the exemplary peptides, tumor cells, cell clones, cell lines, cell supernatants, cell membranes, and/or antigens that are described herein.

“dendritic cell” or “DC” refer to all DCs useful in the present invention, that is, DC is various stages of differentiation, maturation and/or activation. In one embodiment of the present invention, the dendritic cells and responding T cells are derived from healthy volunteers. In another embodiment, the dendritic cells and T cells are derived from patients with cancer or other forms of tumor disease. In yet another embodiment, dendritic cells are used for either autologous or allogeneic application.

“effective amount” refers to a quantity of an antigen or epitope that is sufficient to induce or amplify an immune response against a tumor antigen, e.g., a tumor cell.

“vaccine” refers to compositions that affect the course of the disease by causing an effect on cells of the adaptive immune response, namely, B cells and/or T cells. The effect of vaccines can include, for example, induction of cell mediated immunity or alteration of the response of the T cell to its antigen.

“immunologically effective” refers to an amount of antigen and antigen presenting cells loaded with one or more heat-shocked and/or killed tumor cells that elicit a change in the immune response to prevent or treat a cancer. The amount of antigen-loaded and/or antigen-loaded APCs inserted or reinserted into the patient will vary between individuals depending on many factors. For example, different doses may be required for an effective immune response in a human with a solid tumor or a metastatic tumor.

The terms “nucleic acid” and “oligonucleotide” are used interchangeably herein to mean multiple nucleotides (i.e., molecules comprising a sugar (e.g., ribose or deoxyribose) linked to a phosphate group and to an exchangeable organic base, which is either a substituted pyrimidine (e.g., cytosine (C), thymidine (T) or uracil (U)) or a substituted purine (e.g., adenine (A) or guanine (G)). As used herein, the terms refer to oligodeoxyribonucleotides, oligoribonucleotides (i.e., a polynucleotide minus the phosphate) and any other organic base containing polymer. Nucleic acid molecules can be obtained from existing nucleic acid sources (e.g., genomic or cDNA), but are preferably synthetic (e.g., produced by nucleic acid synthesis).

As used herein, the term “treat”, “treated” or “treating” when used with respect to an infectious disease refers to a prophylactic treatment which increases the resistance of a subject (a subject at risk of infection) to infection with a pathogen, or in other words, decreases the likelihood that the subject will become infected with the pathogen as well as a treatment after the subject (a subject who has been infected) has become infected in order to fight the infection, e.g., reduce or eliminate the infection or prevent it from becoming worse.

The treatment of a subject or with an immunostimulatory oligonucleotide together with checkpoint inhibition or as a means of stimulating checkpoint inhibition is described herein, results in the reduction of infection or the complete abolition of the infection, reduction of the signs/symptoms associated with a disorder associated with a self antigen or the complete abolition on the disorder, or reduction of the signs/symptoms associated with a disorder associated with an addictive substance or the complete abolition of the disorder.

An “antigen” as used herein is a molecule that is capable of provoking an immune response. Antigens include, but are not limited to, cells, cell extracts, proteins, recombinant proteins, purified proteins, polypeptides, peptides, polysaccharides, polysaccharide conjugates, peptide and non-peptide mimics of polysaccharides and other molecules encoded by plasmid DNA, haptens, small molecules, lipids, glycolipids, carbohydrates, whole killed pathogens, viruses and viral extracts, live attenuated virus or viral vector, live attenuated bacteria or a bacterial vector and multicellular organisms such as parasites and allergens. The term antigen broadly includes any type of molecule which is recognized by a host immune system as being foreign or damaged/mutated/overexpressed self proteins.

To practice the invention, the background of PD-L1 and its receptor, PD-1 is provided. Programmed death (PD)-1 was discovered by investigators studying programmed death of T cells. T cell programmed death is very important biologically because it occurs in the thymus, as an essential party of negative selection, as well as in the periphery, when T cells encounter autoantigens. Specifically, two models of T cell death where used. The first was stimulated 2B4.11 (a murine T-cell hybridoma) and the second was interleukin-3 (IL-3)-deprived LyD9 (a murine haematopoietic progenitor cell line). Assuming that common biochemical pathways might be involved in the deaths of 2B4.11 and LyD9, the investigators isolated the PD-1 gene, a novel member of the immunoglobulin gene superfamily, by using subtractive hybridization technique. The predicted PD-1 protein was found to possess a variant form of the consensus sequence found in cytoplasmic tails of signal transducing polypeptides associated with immune recognition receptors. The PD-1 gene demonstrated to be transcribed in both stimulated 2B4.11 and IL-3-deprived LyD9 cells, but not in other death-induced cell lines that did not show the characteristic features of the classical programmed cell death. Expression of the PD-1 mRNA in mouse was restricted to the thymus and increased when thymocyte death was augmented by in vivo injection of anti-CD3 antibody. Further studies utilized an antibody against the murine PD-1 gene product called mAb J43. Analyses by flow cytometry and immunoprecipitation using the antibody revealed that the PD 1 gene product is a 50-55 kDa membrane protein that was expressed on the cell surface of several PD-1 cDNA transfectants and 2B4.11 cells. Since the molecular weight calculated from the amino acid sequence is 29, 310, the PD-1 protein appears to be heavily glycosylated. Normal murine lymphoid tissues such as thymus, spleen, lymph node and bone marrow contained very small numbers of PD-1(+) cells. However, a significant PD-1(+) population appeared in the thymocytes as well as T cells in spleen and lymph nodes by the in vivo anti-CD3 mAb treatment. Furthermore, the PD-1 antigen expression was strongly induced in distinct subsets of thymocytes and spleen T cells by in vitro stimulation with either anti-CD3 mAb or concanavalin A (Con A) which could lead T cells to both activation and cell death. Similarly, PD-1 expression was induced on spleen B cells by in vitro stimulation with anti-IgM antibody. By contrast, PD-1 was not significantly expressed on lymphocytes by treatment with growth factor deprivation, dexamethasone or lipopolysaccharide.

In contrast to murine PD-1, human hPD-1, seems to correlate with activation of T lymphocytes rather than apoptosis. Time-dependent upregulation of hPD-1 mRNA and protein levels in Jurkat cells during phorbol ester (12-O-tetradecanoylphorbol 13-acetate, TPA)-induced differentiation. Human PD-1 protein was also shown to induced during lectin-stimulated activation of human peripheral blood mononuclear cells. Additionally, TPA stimulation of Jurkat cells induces tyrosine phosphorylation of hPD-1, putatively on its cytoplasmic tail signal transduction motif. In order to elucidate mechanisms of T cell inhibition a chimeric molecule consisting of the murine CD28 extracellular domain and human PD-1 cytoplasmic tail. When introduced into CD4 T cells, this construct mimics the activity of endogenous PD-1 in terms of its ability to suppress T cell expansion and cytokine production. The cytoplasmic tail of PD-1 contains two structural motifs, an ITIM and an immunoreceptor tyrosine-based switch motif (ITSM). Mutation of the ITIM had little effect on PD-1 signaling or functional activity. In contrast, mutation of the ITSM abrogated the ability of PD-1 to block cytokine synthesis and to limit T cell expansion. Further biochemical analyses revealed that the ability of PD-1 to block T cell activation correlated with recruitment of Src homology region 2 domain-containing phosphatase-1 (SHP-1) and SHP-2, and not the adaptor Src homology 2 domain-containing molecule 1A, to the ITSM domain. In TCR-stimulated T cells, SHP-2 associated with PD-1, even in the absence of PD-1 engagement. Despite this interaction, the ability of PD-1 to block T cell activation required receptor ligation, suggesting that colocalization of PD-1 with CD3 and/or CD28 may be necessary for inhibition of T cell activation.

Accordingly, by blocking the interaction between PD-1 and PD-L1, immune activation is induced, as well as reduced requirements for costimulation. The invention provides means of breaking tolerance to PD-L-1 by immunization with placental endothelial cells, as well as various derivatives.

The invention provides a therapeutic composition useful for stimulation of immunity against proliferating PD-L1 and thus augmenting immunity. In one specific embodiment of the invention, endothelial cells are derived from placental tissue, isolated into a homogeneous or semi-homogeneous mixture, treated with agents capable of augmenting immunogenicity, and subsequently administered into a recipient in which immune response to proliferating endothelium is desired. In one specific example, endothelial cells are purified from a human placenta according to the following steps: a) Fetal membranes are manually peeled back and the villous tissue is isolated from the placental structure, with caution being used not to extract the deciduas or fibrous elements of the placental structure; b) The fetal villous tissue is subsequently washed with cold saline to remove blood and scissors are used to mechanically digest the tissue into pieces as small as possible; c) The minced tissue is then enzymatically digested. Specifically, about 25 grams of minced tissue is incubated with approximately 56 ml of liquid solution which has been pre-warmed to a temperature of 37 Celsius. Said solution comprised of Hanks Buffered Saline Solution (HBSS) supplemented with 25 mM of HEPES and containing Calcium and Magnesium, said solution containing 0.28% collagenase, 0.25% dispase, and 0.01% DNAse (added during the incubation periods as described below); d) The mixture of minced placental villus tissue and digesting solution is incubated under stirring conditions for three incubation periods of 20 minutes each. Ten minutes after the first incubation period and immediately after the second and third incubation periods, the DNAse is added to make up a total concentration of DNase, by volume, of 0.01%; e) In the first and second incubations, the incubation flask is set at an angle, and the tissue fragments are allowed to settle for approximately 1 minute, with 35 ml of the supernantant cell suspension being collected and replaced by 38 ml (after the first digestion) or 28 ml (after the second digestion) of fresh digestion solution. After the third digestion the whole supernatant is collected; f) The supernatant collected from all three incubations is pooled and is poured through approximately four layers of sterile gauze and through one layer of 70 micro meter polyester mesh. The filtered solution is then centrifuged for 1000 g for 10 minutes through diluted new born calf serum, said new born calf serum diluted at a ratio of 1 volume saline to 7 volumes of new born calf serum; g) The pooled pellet is then resuspended in 35 ml of warm DMEM with 25 mM HEPES containing 5 mg DNase I; h) The suspension is then mixed with 10 ml of 90% Percoll to give a final density of 1.027 g/ml and is centrifuged at 550 g for 10 minutes with the centrifuge brake off; i) The pellet is then collected and resuspended in 15 ml of DMEM with 25 mM HEPES that is layered over a discontinuous Percoll gradient comprising of 20%-70% Percoll in 10% steps and centrifuged at 1900 g for 20 minutes; j) The cells found at the 1.037 g/ml and 1.048 g/ml are collected utilized for the generation of a cellular vaccine product.

Said cellular vaccine product from step “j”, in a preferred embodiment is treated with an agent capable of augmenting immunogenicity. Said immunogenicity in this context refers to ability to enhance recognition by recipient immune system. In one embodiment, immunogenicity refers to enhanced expression of HLA I and/or HLA II molecules. In another embodiment, immunogenicity refers to enhanced expression of costimulatory molecules. Said costimulatory molecules are selected from a group comprising of: CD27; CD80; CD86; ICOS; OX-4; and 4-1 BB. In another embodiment, immunogenicity refers to enhanced ability to stimulate proliferation of allogeneic lymphocytes in a mixed lymphocyte reaction. Immunogenicity may be augmented by incubation with one of the lymphokine or cytokine proteins that are known in the art, or with a member of the interferon family. In one particular embodiment, said purified endothelial cells are incubated with interferon gamma. In one particular embodiment, interferon gamma is incubated with endothelial cells, whether purified or unpurified for a period of approximately 48 hours, at a concentration of approximately 150 IU/ml. Endothelial cells may be expanded after purification as described above before treatment with agents capable of augmenting immunogenicity. For example, endothelial cells may be treated with an endothelial cell mitogen. Said endothelial cell mitogen may be any protein, polypeptide, variant or portion thereof that is capable of, directly or indirectly, inducing endothelial cell growth. Such proteins include, for example, acidic and basic fibroblast growth factors (aFGF) (GenBank Accession No. NP.sub.—149127) and bFGF (GenBank Accession No. AAA52448), vascular endothelial growth factor (VEGF) (GenBank Accession No. AAA35789 or NP.sub.—001020539), epidermal growth factor (EGF) (GenBank Accession No. NP.sub.—001954), transforming growth factor .alpha. (TGF-.alpha.) (GenBank Accession No. NP.sub.—003227) and transforming growth factor beta (TFG-beta) (GenBank Accession No. 1109243A), platelet-derived endothelial cell growth factor (PD-ECGF) (GenBank Accession No. NP.sub.—001944), platelet-derived growth factor (PDGF) (GenBank Accession No. 1109245A), tumor necrosis factor alpha (TNF-alpha) (GenBank Accession No. CAA26669), hepatocyte growth factor (HGF) (GenBank Accession No. BAA14348), insulin like growth factor (IGF) (GenBank Accession No. P08833), erythropoietin (GenBank Accession No. P01588), colony stimulating factor (CSF), macrophage-CSF (M-CSF) (GenBank Accession No. AAB59527), granulocyte/macrophage CSF (GM-CSF) (GenBank Accession No. NP.sub.—000749), monocyte chemotactic protein-1 (GenBank Accession No. P13500) and nitric oxide synthase (NOS) (GenBank Accession No. AAA36365). See, Klagsbrun, et al., Annu. Rev. Physiol., 53:217-239 (1991); Folkman, et al., J. Biol. Chem., 267:10931-10934 (1992) and Symes, et al., Current Opinion in Lipidology, 5:305-312 (1994). Variants or fragments of a mitogen may be used as long as they induce or promote endothelial cell or endothelial progenitor cell growth. Preferably, the endothelial cell mitogen contains a secretory signal sequence that facilitates secretion of the protein. Proteins having native signal sequences, e.g., VEGF, are preferred. Proteins that do not have native signal sequences, e.g., bFGF, can be modified to contain such sequences using routine genetic manipulation techniques. See, Nabel et al., Nature, 362:844 (1993). Before expansion, endothelial cells may be further purified based on expression of surface receptors using affinity-based methodologies that are known to one of skill in the art, said methodologies include magnetic activated cell sorting (MACS), cell panning, or affinity chromatography. Other methodologies such as fluorescent activated cell sorting (FACS) may also be used. Various lectins are known to have selectivity to endothelial cells, for example, Ulex europaeus agglutinin I is known to possess ability to bind to endothelial cells and endothelial progenitor cells. It is within the scope of the current invention to define “endothelial cell” as including “endothelial progenitor cell.”

The cancer vaccine formulation may be utilized in conjunction with known adjuvants in order to induce an immune response that is Th1 or Th17-like, and which will inhibit the activity of PD-L1 in the recipient. Such adjuvant compounds are known in the art to boost the activity of the immune system and are now under study as possible adjuvants, particularly for vaccine therapies. Some of the most commonly studied adjuvants are listed below, but many more are under development. For example, Levamisole, a drug originally used against parasitic infections, has recently been found to improve survival rates among people with colorectal cancer when used together with some chemotherapy drugs. It is often used as an immunotherapy adjuvant because it can activate T lymphocytes. Additionally, the compound has been demonstrated to induce maturation of dendritic cells, further supporting an immune modulatory role. Levamisole is now used routinely for people with some stages of colorectal cancer and is being tested in clinical trials as a treatment for other types of cancer. Additionally, it has been shown to augment efficacy of other immunotherapeutic agents such as interferon. Aluminum hydroxide (alum) is one of the most common adjuvants used in clinical trials for cancer vaccines. It is already used in vaccines against several infectious agents, including the hepatitis B virus. Bacille Calmette-Guerin (BCG) is a bacterium that is related to the bacterium that causes tuberculosis. The effect of BCG infection on the immune system makes this bacterium useful as a form of anticancer immunotherapy. BCG was one of the earliest immunotherapies used against cancer, either alone, or in combination with other therapies such as hormonal, chemotherapy or radiotherapy. It is FDA approved as a routine treatment for superficial bladder cancer. Its usefulness in other cancers as a nonspecific adjuvant is also being tested or has demonstrated therapeutic effects. Researchers are looking at injecting BCG to give an added stimuli to the immune system when using chemotherapy, radiation therapy, or other types of immunotherapy. Thus in various embodiments of the current invention, one of skill in the art is directed towards references which have utilized BCG as an adjuvant for other therapies for concentrations and dosing regimens that would apply to the current invention for elicitation of immunity towards proliferating endothelial cells. Incomplete Freund's Adjuvant (IFA) is given together with some experimental therapies to help stimulate the immune system and to increase the immune response to cancer vaccines, both protein and peptide in part by providing a localization factor for T cells. IFA is a liquid consisting of an emulsifier in white mineral oil. Another vaccine adjuvant useful for the present invention is interferon alpha, which has been demonstrated to augment NK cell activity, as well as to promote T cell activation and survival. QS-21 is a relatively new immune stimulant made from a plant extract that increases the immune response to vaccines used against melanoma. DETOX is another relatively new adjuvant. It is made from parts of the cell walls of bacteria and a kind of fat. It is used with various immunotherapies to stimulate the immune system. Keyhole limpet hemocyanin (KLH) is another adjuvant used to boost the effectiveness of cancer vaccine therapies. It is extracted from a type of sea mollusc. Dinitrophenyl (DNP) is a hapten/small molecule that can attach to tumor antigens and cause an enhanced immune response. It is used to modify tumor cells in certain cancer vaccines.

In one embodiment of the invention proliferating endothelial cells treated with an agent to stimulate immunogenicity are lysed and protein extracts are extracted and utilized as a vaccine. In some embodiments specific immunogenic peptides may be isolated for said cell lysate. In other embodiments, lyophilization of endothelial cells is performed subsequent to treatment with an agent that augments immunogenicity. In embodiments utilizing cellular extracts, various formulations may be generated. The formulations may conveniently be presented in unit dosage form and may be prepared by any of the methods well known in the art of pharmacy. Such methods include the step of bringing into association the active ingredient (for an antigenic molecule, construct or chimaeric polypeptide of the invention) with the carrier which constitutes one or more accessory ingredients. In general the formulations are prepared by uniformly and intimately bringing into association the active ingredient with liquid carriers or finely divided solid carriers or both, and then, if necessary, shaping the product.

In one embodiment of the invention, said ValloVax™ (Ichim et al, J Transl Med 2015) induces increased permeability of tumor endothelium allow for increased efficacy of chemotherapy and radiotherapy.

Additionally, given that tumor endothelium expresses immune killing molecules such as Fas ligand, in one embodiment, the use of ValloVax™ together with immunotherapy is disclosed.

Formulations in accordance with the present invention suitable for oral administration may be presented as discrete units such as capsules, cachets or tablets, each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste. In situations where an orally available vaccine is desirable, a tablet may be made by compression or moulding, optionally with one or more accessory ingredients. Compressed tablets may be prepared by compressing in a suitable machine the active ingredient in a free-flowing form such as a powder or granules, optionally mixed with a binder (eg povidone, gelatin, hydroxypropylmethyl cellulose), lubricant, inert diluent, preservative, disintegrant (eg sodium starch glycolate, cross-linked povidone, cross-linked sodium carboxymethyl cellulose), surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethylcellulose in varying proportions to provide desired release profile. Formulations suitable for topical administration in the mouth include lozenges comprising the active ingredient in a flavoured base, usually sucrose and acacia or tragacanth; pastilles comprising the active ingredient in an inert base such as gelatin and glycerin, or sucrose and acacia; and mouth-washes comprising the active ingredient in a suitable liquid carrier. Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described. Nasal sprays may be useful formulations. Preferred unit dosage formulations are those containing a single or daily dose or unit, daily sub-dose or an appropriate fraction thereof, of an active ingredient.

It will be appreciated that the therapeutic molecule can be delivered to the locus by any means appropriate for localized administration of a drug. For example, a solution of the therapeutic molecule can be injected directly to the site or can be delivered by infusion using an infusion pump. The construct, for example, also can be incorporated into an implantable device which when placed at the desired site, permits the construct to be released into the surrounding locus. The therapeutic molecule may be administered via a hydrogel material. The hydrogel is non-inflammatory and biodegradable. Many such materials now are known, including those made from natural and synthetic polymers. In a preferred embodiment, the method exploits a hydrogel which is liquid below body temperature but gels to form a shape-retaining semisolid hydrogel at or near body temperature. Preferred hydrogel are polymers of ethylene oxide-propylene oxide repeating units. The properties of the polymer are dependent on the molecular weight of the polymer and the relative percentage of polyethylene oxide and polypropylene oxide in the polymer. Preferred hydrogels contain from about 10% to about 80% by weight ethylene oxide and from about 20% to about 90% by weight propylene oxide. A particularly preferred hydrogel contains about 70% polyethylene oxide and 30% polypropylene oxide. Hydrogels which can be used are available, for example, from BASF Corp., Parsippany, N.J., under the tradename Pluronic.sup.R. Although subcutaneous, intradermal, and intramuscular routes of administration are preferred, administration into lymphatics of the vaccine preparation is also envisioned within the scope of the current invention. Endpoints guiding the practitioner of the invention include: a) ability of the vaccine to stimulate immunity towards proliferating endothelial cells; b) ability of the vaccine to stimulate immunity towards cancer-associated molecules; and c) ability of the vaccine to stimulate immunity towards tumor cells.

In one embodiment the invention provides a means of generating a population of cells with ability to inhibit endothelial cell proliferation. In one embodiment approximately 50 ml of peripheral blood is extracted from a cancer patient and peripheral blood monoclear cells (PBMC) are isolated using the Ficoll Method. PBMC are subsequently resuspended in approximately 10 ml RPMI media with 10% fetal calf serum and allowed to adhere onto a plastic surface for 2-4 hours. The adherent cells are then cultured at 37° C. in RPMI media supplemented with 1,000 U/mL granulocyte-monocyte colony-stimulating factor and 500 U/mL IL-4. This procedure, or a procedure similar to it, can be utilized for the generation of dendritic cells. Half of the volume of the GM-CSF and IL-4 supplemented media is changed every other day. Immature DCs are harvested on day 7. In one embodiment said generated DC are treated with endothelial cell extracts isolated from placental or otherwise proliferating endothelial cells. Said extracts are added to said immature dendritic cells on day 7. Endothelial pulsed dendritic cells may be administered directly as a vaccine, or may be utilized to stimulate autologous patient T cell clones in vitro. Said T cell clones may be selected for specificity to proliferating endothelial cells. Additionally, in some embodiments, whether for in vitro stimulation of T cells, or for direct use as a tumor vaccine, the endothelial cell pulsed dendritic cells may be further purified from culture through use of flow cytometry sorting or magnetic activated cell sorting (MACS), or may be utilized as a semi-pure population. In one embodiment DC are exposed to agents capable of stimulating maturation in vitro subsequent to pulsing with endothelial cell extracts. Specific means of stimulating in vitro maturation include culturing DC or DC containing populations with a toll like receptor agonist. Another means of achieving DC maturation involves exposure of DC to TNF-alpha at a concentration of approximately 20 ng/mL. In another embodiment, a mixture of endothelial cells together with immature dendritic cells is used as a combination cellular vaccine. In another embodiment, endothelial cells (live or extracts or fixed) are administered in combination with dendritic cells together with activated T cells and/or NK cells. In order to activate T cells and/or NK cells in vitro, cells are cultured in media containing approximately 1000 IU/ml of interferon gamma. Incubation with interferon gamma may be performed for the period of 1 hour to the period of 14 days. Preferably, incubation is performed for approximately 48 hours, after which T cells and/or NK cells may be further stimulated via the CD3 and CD28 receptors. One means of accomplishing this is by addition of antibodies capable of activating these receptors. In one embodiment approximately, 3 ug/ml of anti-CD3 antibody is added, together with approximately 2 ug/ml anti-CD28. In order to promote survival of T cells and NK cells, was well as to stimulate proliferation, a T cell/NK mitogen may be used. In one embodiment the cytokine IL-2 is utilized. Specific concentrations of IL-2 useful for the practice of the invention are approximately 400 u/mL IL-2. Media containing IL-2 and antibodies may be changed every two days for approximately 7-24 days. In one particular embodiment DC are included to said T cells and/or NK cells in order to endow cytotoxic activity towards tumor cells. In a particular embodiment, inhibitors of caspases are added in the culture so as to reduce rate of apoptosis of T cells and/or NK cells. Generated cells can be administered to a subject intradermally, intramuscularly, subcutaneously, intraperitoneally, intraarterially, intravenously (including a method performed by an indwelling catheter), intratumorally, or intralymphatically.

In some embodiments, endothelial cells are increased in immunogenicity by culture with T cells that are autologous or allogeneic to the donor of said endothelial cells. Said T cells may be activated by their allogeneic interaction with said endothelial cells, or may be introduced into contact with endothelial cells in an already preactivated state. In order to preactive T cells, firstly lymphocytes are collected and separation into the T cell population and cell sub-population containing a T cell can be performed, for example, by fractionation of a mononuclear cell fraction by density gradient centrifugation, or a separation means using the surface marker of the T cell as an index of detection. Subsequently, isolation based on surface markers may be performed. Examples of the surface marker include CD2, CD3, CD8 and CD4, and separation methods depending on these surface markers are known to one of skill in the art. For example, the step can be performed by mixing a carrier such as beads or a culturing flask onto which an anti-CD8 antibody has been immobilized (cell panning), with a cell population containing a T cell, and recovering a CD8-positive T cell bound to the carrier. As the beads on which an anti-CD8 antibody has been immobilized, for example, CD8 MicroBeads), Dynabeads M450 CD8, and Eligix anti-CD8 mAb coated nickel particles can be suitably used. This is also the same as in implementation using CD4 as marker of detection and, for example, CD4 MicroBeads, Dynabeads M-450 CD4 can also be used. In some embodiments of the invention, T regulatory cells are depleted before initiation of the culture, with the idea of “derepressing” suppressive elements within the heterogeneous T cell population. Depletion of T regulatory cells may be performed by negative selection by removing cells that express makers such as neuropilin, CD25, CD4, CD105, CTLA4, and membrane bound TGF-beta. Experimentation by one of skill in the art may be performed with different culture conditions in order to generate effector lymphocytes, or cytotoxic cells, that possess both maximal activity in terms of tumor killing, as well as migration to the site of the tumor. For example, the step of culturing the cell population and cell sub-population containing a T cell can be performed by selecting suitable known culturing conditions depending on the cell population. In addition, in the step of stimulating the cell population, known proteins and chemical ingredients, etc., may be added to the medium to perform culturing. For example, cytokines, chemokines or other ingredients may be added to the medium. Herein, the cytokine is not particularly limited as far as it can act on the T cell, and examples thereof include IL-2, IFN-gamma, IL-15, IL-7, IFN-alpha, IL-12, CD40L, and IL-27. From the viewpoint of enhancing cellular immunity, particularly suitably, IL-2, IFN-gamma, or IL-12 is used and, from the viewpoint of improvement in survival of a transferred T cell in vivo, IL-7, IL-15 or IL-21 is suitably used. In addition, the chemokine is not particularly limited as far as it acts on the T cell and exhibits migration activity, and examples thereof include RANTES, CCL21, MIP1 alpha, MIP1 beta, CCL19, CXCL12, IP-10 and MIG. The stimulation of the cell population can be performed by the presence of a ligand for a molecule present on the surface of the T cell, for example, CD3, CD28, or CD44 and/or an antibody to the molecule. Further, the cell population can be stimulated by contacting with other lymphocytes or antigen presenting cells (dendritic cell) presenting a target peptide such as a peptide derived from an endothelial cell antigen. In addition to assessing cytotoxicity and migration as end points, it is within the scope of the current invention to optimize the cellular product based on other means of assessing T cell activity, for example, the function enhancement of the T cell in the method of the present invention can be assessed at a plurality of time points before and after each step using a cytokine assay, an antigen-specific cell assay such as the tetramer assay, a proliferation assay, a cytolytic cell assay, or an in vivo delayed hypersensitivity test using a recombinant endothelial cell-associated antigen or an immunogenic fragment or an antigen-derived peptide. In a preferred embodiment, the antigen derived peptides are specifically associated with proliferating endothelial cells, such as endothelial cells found in proximity to the tumor. Examples of an additional method for measuring an increase in an immune response include a delayed hypersensitivity test, flow cytometry using a peptide major histocompatibility gene complex tetramer. a lymphocyte proliferation assay, an enzyme-linked immunosorbent assay, an enzyme-linked immunospot assay, cytokine flow cytometry, a direct cytotoxity assay, measurement of cytokine mRNA by a quantitative reverse transcriptase polymerase chain reaction, or an assay which is currently used for measuring a T cell response such as a limiting dilution method. In vivo assessment of the efficacy of the generated cells using the invention may be assessed in a living body before first administration of the T cell with enhanced function of the present invention, or at various time points after initiation of treatment, using an antigen-specific cell assay, a proliferation assay, a cytolytic cell assay, or an in vivo delayed hypersensitivity test using a recombinant endothelial-associated antigen or an immunogenic fragment or an antigen-derived peptide. Examples of an additional method for measuring an increase in an immune response include a delayed hypersensitivity test, flow cytometry using a peptide major histocompatibility gene complex tetramer. a lymphocyte proliferation assay, an enzyme-linked immunosorbent assay, an enzyme-linked immunospot assay (ELISPOT), cytokine flow cytometry, a direct cytotoxity assay, measurement of cytokine mRNA by a quantitative reverse transcriptase polymerase chain reaction, or an assay which is currently used for measuring a T cell response such as a limiting dilution method. Further, an immune response can be assessed by a weight, diameter or malignant degree of a tumor possessed by a living body, or the survival rate or survival term of a subject or group of subjects.

### EXAMPLE

### Stimulation of Anti PD 1L Antibody by Vallovax™ Vaccination

Patient 1 was a 55-year old male diagnosed with colon cancer and adjacent lymphadenopathy who had undergone colon resection and resection of extraperitoeum metastases. As per the standard protocol for ValloVax™ administration, Patient 1 was treated with three subcutaneous injections of ValloVax™ on days 0, 7 and 14 and serum collection was performed pre-immunization and 12 weeks and 22 weeks for detection of antibodies against tumor endothelial cell antigens in serum. The analysis for detection of antibodies against tumor endothelial antigens were conducted prior to immunization, and four and ten weeks following immunization. Patient antibody analyses were conducted using ELISA techniques whereby plates were incubated in microwell plates with their respective capture proteins and dilutions of patient serum samples. The assay was performed using an OPD developing agent and optical density (OD) readings at 490 nm.

Patient 2, a 48-year old female, presented with rectal ulcerated moderately differentiated adenocarcinoma and a previous history of diffuse large non-B cell non-Hodgkins lymphoma. The patient was also treated with three subcutaneous injections of ValloVax™ on days 0, 7 and 14. Quantitation of antibodies against tumor endothelial antigens was performed using serum samples harvested at 10 weeks and 16 weeks post-immunization to compare to pre-immunization serum.

Patient 3 was a 44-year old female with recurrent hemangioblastoma who previously underwent stereotactic radiosurgery and was administered ValloVax™ on days 0, 7 and 14. Serum samples taken pre-immunization were compared to those taken at one timepoint that was 7 weeks post-vaccination.

1. A method of inducing antibodies to PD-L1 in a mammal comprising the steps of:
a) selecting a placental endothelial cell population;
b) treating said population with an agent capable of enhancing antigenicity of said placental endothelial cell population; and
c) administering said placental endothelial cell population in a mammal in a manner, concentration, and frequency capable of stimulating antibody responses to said PD-L1.

2. The method of claim 1, wherein said placental endothelial cell population expresses the marker CD31.

3. The method of claim 1, wherein said placental endothelial cell population expresses markers selected from a group comprising of:
a) TEM-1;
b) ROBO-4;
c) ROBO 1-18;
d) VEGFR2;
e) CD109;
f) survivin; and
g) CD93.

4. The method of claim 1, wherein said enhancement of immunogenicity is accomplished by pretreated with an agent capable of augmenting immunogenicity of said cellular immunogens.

5. The method of claim 4, wherein said agents capable of augmenting immunogenicity increase expression of an HLA or HLA-like molecule.

6. The method claim 4, wherein said agents capable of augmenting immunogenicity increase expression of costimulatory molecules.

7. The method of claim 6, wherein said costimulatory molecules are selected from a group comprising of:
a) CD40;
b) CD 80;
c) CD86;
d) OX40;
e) ICOS; and
f) 4-1 BB.

8. The method of claim 4 wherein said agents capable of augmenting immunogenicity are selected from a group comprising of:
a) IL-1;
b) IL-2;
c) TNF-alpha;
d) IFN-gamma;
e) IL-33; and
f) IL-27.

9. The method of claim 4, wherein augmentation of immunogenicity is achieved by exposure of said cells to sublethal hyperthermia.

10. The method of claim 9, wherein said sublethal hyperthermia is sufficient to augment expression of heat shockproteins in said cell.

11. The method of claim 10, wherein said heat shock proteins are selected from a group comprising of:
a) gp96;
b) hsp 35;
c) hsp 70; and
d) hsp 95.

12. The method of claim 1, wherein said placental endothelial cells are endothelial progenitor cell.

13. The method of claim 12, wherein said endothelial progenitor cells are purified from a source selected from a group comprising of:
a) cord blood endothelial progenitor cells;
b) circulating endothelial progenitor cells;
c) bone marrow endothelial progenitor cells; and
d) placental matrix endothelial progenitor cells.

14. The method of claim 13, wherein said endothelial progenitor cells are capable of forming endothelial colonies when cultured in a matrigel substrate.

15. The method of claim 13, wherein said endothelial progenitor cells are capable of forming endothelial colonies when cultured in a methylcellulose substrate.

16. The method of claim 13, wherein said endothelial progenitor cells are capable of forming blood vessel-like tubes when implanted in an immune deficient mouse.

17. The method of claim 13, wherein said endothelial progenitor cells are in a proliferative state.

18. The method of claim 13, wherein said proliferative state of said endothelial progenitor cells is assessed by expression of PCNA.

19. The method of claim 12, wherein said endothelial progenitor cells express the marker CD34.

20. The method of claim 2, wherein said endothelial progenitor cells express the marker CD31.

21. The method of claim 2, wherein said endothelial progenitor cells express the marker CD164.

22. The method of claim 2, wherein said endothelial progenitor cells express the marker CD117.

23. The method of claim 2, wherein said endothelial progenitor cells are cultured under conditions resembling the tumor microenvironment in order to endow a tumor endothelial-like phenotype on said endothelial progenitor cells.

24. The method of claim 23, wherein inflammatory agents are administered at concentrations similar to those found in tumors in order to elicit a tumor endothelial-like phenotype onto said endothelial progenitor cells.

25. The method of claim 24, wherein said inflammatory agents are selected from a group comprising of:
a) IL-1,
b) TNF-alpha;
c) IL-6; and
d) IL-33.

26. The method of claim 23, wherein said tumor microenvironment is replicated by culturing cells in conditions of hypoxia.

27. The method of claim 23, wherein said tumor microenvironment is replicated by culturing cells in conditions of acidosis.

28. The method of claim 23, wherein said tumor microenvironment is replicated by culturing cells in conditions of high lactic acid.
